(19) United States
(12) Patent Application Publication
(10) Pub. No.: US 2023/0212642 A1
(43) Pub. Date: Jul. 6, 2023

(54) METHOD AND SYSTEM FOR NON-MICROBIAL PREDICTION OF ANTIMICROBIAL EFFICACY OF AN ANTIMICROBIAL ITEM

(71) Applicants: William Alexander ANDERSON, Waterloo (CA); Shazia TANVIR, Waterloo (CA)

(72) Inventors: William Alexander ANDERSON, Waterloo (CA); Shazia TANVIR, Waterloo (CA)

(21) Appl. No.: 17/927,404

(22) PCT Filed: May 27, 2021
(86) PCT No.: PCT/CA2021/050719

§ 371 Date: Nov. 23, 2022

Related U.S. Application Data
(60) Provisional application No. 63/102,002, filed on May 27, 2020.

(51) Int. Cl. C12Q 1/18

(52) U.S. Cl. CPC C12Q 1/18

(57) ABSTRACT

The disclosure is directed at a system and method for non-microbial prediction of antimicrobial efficacy of an antimicrobial item, such as, a metal surface or materials impregnated with biocidal metal ions or nanoparticles. A test formulation is applied to a surface of interest and a color change of the test formulation is then analyzed and then correlated with antimicrobial activity.

### CROSS-REFERENCE TO OTHER APPLICATIONS

The current disclosure claims priority from U.S. Provisional Application No. 63/102,002 filed May 27, 2020 which is hereby incorporated by reference.

### FIELD

The disclosure is generally directed at antimicrobial efficacy testing, and, more specifically, at a method and system for non-microbial prediction of antimicrobial efficacy of an antimicrobial item.

### BACKGROUND

Typically, as a part of a product development process, when a new item includes a surface that is engineered with an antimicrobial agent, samples are sent out for the third-party verification of the efficiency, or efficacy, of the antimicrobial activity. Normally, this kind of validation may take weeks or months to get back to the manufacturers, sometimes with negative feedback.

Currently, this testing requires a tester to apply test microbes to the material or surface and then to recover, culture and count the microbes. Typically, this requires the preparation of a microbial culture under controlled conditions, followed by application of some of the culture to the surface or material to be tested and allowing contact for a fixed period of time. The culture is then recovered from the surface or material, then serially diluted and applied to agar plates (for bacteria and fungi). These plates are then incubated at controlled conditions for a period of 24 to 48 hours, typically. The number of surviving colonies on the agar plates are counted, and these counts are compared to the initial culture and control (non-antimicrobial) tests to determine the extent of antimicrobial activity of the surface or material.

The current procedure is very time consuming as it requires time to transport the item to a lab and then to culture or grow the microbes to determine the antimicrobial efficacy of the material or surface from which the sample was taken.

There is no simple test currently available that could help these companies quickly understand the level of antimicrobial efficacy of a new item. This bottleneck and blind spot in their work could cause them a significant financial and time loss.

Therefore, there is provided a novel method and system for non-microbial determination of antimicrobial efficacy.

### SUMMARY

The disclosure is directed at a method and system for non-microbial prediction of antimicrobial efficacy. More specifically, the disclosure is directed at determining antimicrobial efficacy of copper, zinc, zinc oxide, titanium dioxide, or silver-based surfaces and materials.

The disclosure is directed at methods and system for rapid testing of copper, zinc, zinc oxide, titanium dioxide or silver released from antimicrobial items, such as, but not limited to metal surfaces or materials impregnated with biocidal metal ions or nanoparticles. The method and system of the disclosure are based on chemical transformations that result in a color change. This color change happens within a few minutes and can be detected either visually or with the use of spectrophotometer or colorimeter instruments. Furthermore, this color change can be correlated with the antimicrobial activity of the treated surface or material such as by comparing the color change with a predetermined chart or by comparing the color change with an expected color change.

In one embodiment, the disclosure is directed at a system and method for the rapid testing of copper, zinc, zinc oxide, titanium dioxide or silver ions and reactive oxygen species released from surfaces and treated materials that may be used for validation of antimicrobial activity of the treated surface or material. In an embodiment, a test solution or formulation is applied to the surface or treated material which may result in a visible color change of the test solution. This color change may occur in a few minutes and can be detected either visually or with the use of spectrophotometer or colorimeter instruments.

An advantage of the current disclosure is that users may rapidly assess whether the material of interest has significant antimicrobial activity, without directly resorting to very time-consuming and expensive microbial laboratory work that can require multiple days for test results.

In one aspect of the disclosure, there is provided a method for non-microbial prediction of antimicrobial efficacy of an antimicrobial item including producing a test solution; applying the test solution to the antimicrobial item; analyzing a color change of the test solution after exposure of the test solution to the antimicrobial item; and correlating the color change with a level of antimicrobial efficacy.

In another aspect, the test solution reacts with a leaching of metal from the antimicrobial item. In a further aspect, producing a test solution includes mixing bicinchoninic acid (BCA) ascorbic acid, sodium bicarbonate and sodium phosphate dibasic. In yet another aspect, the antimicrobial item includes a copper surface, copper based alloys, nanoparticle and ions composites.

In a further aspect, producing a test solution includes mixing resazurin with a liquid. In an aspect, mixing resazurin with a liquid includes mixing resazurin with water. In yet another aspect, the antimicrobial item includes a zinc surface.

In another aspect, producing a test solution comprises mixing, silver nitrate, trisodium citrate, sodium borohydride and hydrogen peroxide to create silver nanoparticles. In a further aspect, the silver nanoparticles are silver nanoprisms. In yet another aspect, the antimicrobial item includes a silver surface.

In another aspect of the disclosure, there is provided a test material for use in non-microbial prediction of antimicrobial efficacy of an antimicrobial item including a component that changes color when in contact with metal ions leaching from the antimicrobial item or reactive oxygen species on the antimicrobial item.

In a further aspect, the component includes bicinchoninic acid (BCA). In yet another aspect, the test material further includes a weak reducing agent. In another aspect, the test material further includes at least one of ascorbic acid, sodium bicarbonate, sodium phosphate dibasic, dextrose and poly(vinyl alcohol).

In another aspect, the component includes a redox dye. In a further aspect the redox dye includes one of resazurin, 2,3-Bis-(2-Methoxy-4-Nitro-5-Sulfophenyl)-2H-Tetrazolium-5-Carboxanilide (XTT), nitro blue tetrazolium (NBT), 3-(4,5-dimethylthiazol-2-yl)-2,5-diphenyltetrazolium bromide (MTT) or 5-Cyano-2,3-ditolyl tetrazolium chloride (CTC).

In an aspect, the component includes citrate capped silver nanoparticles, PVP capped nanoparticles, citrate capped gold nanoparticles or PVP capped gold nanoparticles. In another aspect, the silver nanoparticles include prismatic silver nanoparticles.

### BRIEF DESCRIPTION OF THE DRAWINGS

The foregoing and other features and advantages of the disclosure will be apparent from the following description of embodiments thereof as illustrated in the accompanying drawings. The accompanying drawings, which are incorporated herein and form a part of the specification, further serve to explain the principles of the disclosure and to enable a person skilled in the pertinent art to make and use the invention. The drawings are not to scale.

FIG. 1a is a schematic diagram of a method of non-microbial prediction of anti-microbial efficacy of an antimicrobial item;

FIG. 1b is a chart showing different apparatus for non-microbial prediction of anti-microbial efficacy of an antimicrobial item;

FIG. 2a is a schematic diagram of a system for non-microbial prediction of anti-microbial efficacy of a copper surface;

FIG. 2b is a schematic diagram of another embodiment of a system for non-microbial prediction of anti-microbial efficacy of a copper surface;

FIG. 3a is a chart showing a quantification of copper¹⁺-BCA using spectrophotometry;

FIG. 3b is a calibration curve;

FIG. 4 is a chart showing copper ion release from different surfaces versus antimicrobial performance using a bacterium;

FIG. 5 are photos showing results from testing using a copper test formulation;

FIG. 6 are photos showing surface imprinting results using a copper test formulation;

FIG. 7a is a chart showing copper ion release using a spectrophotometer;

FIG. 7b is a chart showing absorbance of copper ions;

FIG. 8a is a schematic diagram of a system for non-microbial prediction of anti-microbial efficacy of a zinc surface;

FIG. 8b is a schematic diagram of another embodiment of a system for non-microbial prediction of anti-microbial efficacy of a zinc surface;

FIG. 9a is a chart showing a whole spectral scan of resazurin reduction by zinc;

FIG. 9b is a chart showing an evolution of a resazurin peak at 600 nm with respect to resazurin reduction by zinc;

FIG. 10a is a chart showing a whole spectral scan of the photocatalytic conversion of resazurin;

FIG. 10b is a chart showing an evolution of a resazurin peak at 600 nm with respect to the photocatalytic conversion of resazurin;

FIG. 11 is a chart showing absorption of prismatic nanoparticles in a solution;

FIG. 12 is a TEM image of prismatic nanoparticles;

FIG. 13 is a chart showing an elemental analysis of prismatic nanoparticles;

FIG. 14 is a chart showing an elemental analysis of spherical nanoparticles;

FIG. 15 is a chart showing surface zeta potential of spherical nanoparticles coated with different materials;

FIG. 15b is a chart showing surface zeta potential of spherical nanoparticles coated with different materials;

FIG. 16a is a chart showing a visible spectrum a surface tested with a silver test formulation; and

FIG. 16b is a chart showing a calibration curve for the spectrum of FIG. 16a.

### DETAILED DESCRIPTION

The disclosure is directed at a method and system for non-microbial, or chemical, determination or indication of antimicrobial efficacy of an antimicrobial item. Examples of antimicrobial items include, but are not limited to, a metal surface or materials impregnated with biocidal metal ions or nanoparticles. In one embodiment, the disclosure provides a non-microbial determination of the antimicrobial efficacy of surfaces or materials that have been impregnated or combined with one of copper, zinc, zinc oxide, titanium dioxide or silver. In another embodiment, the disclosure provides non-microbial determination of the antimicrobial efficacy of photocatalytic surfaces or materials.

One advantage of the disclosure is the provision of a method and system for testing antimicrobial efficacy of an antimicrobial item that is faster than current techniques. Another advantage of the disclosure is that the disclosure may be performed without a high level of training. Another advantage of the current disclosure is that, for some embodiments, this testing may be performed without the use of any sophisticated equipment.

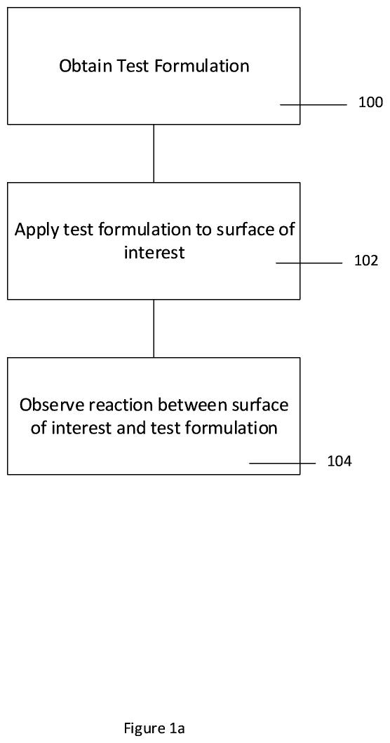

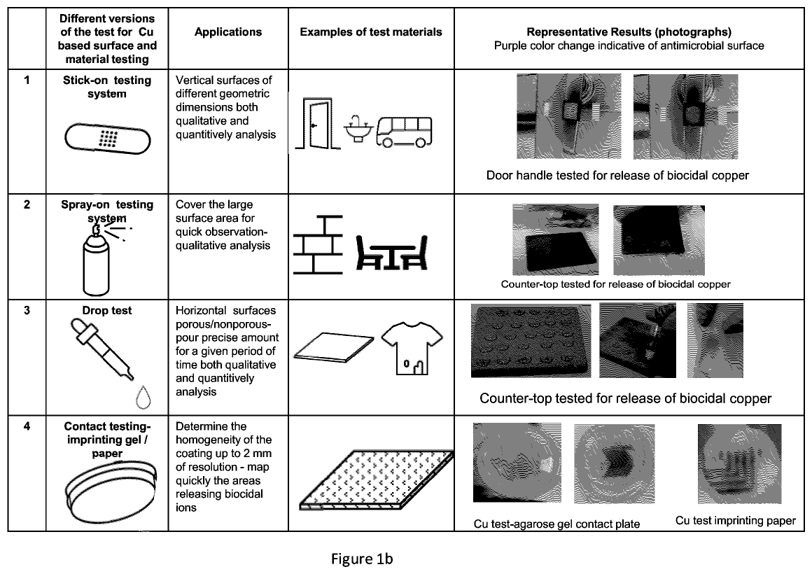

Turning to FIG. 1, a method of non-bacterial, or chemical, determination of antimicrobial efficacy is shown. Initially, a test formulation is obtained (100). In one embodiment, the test formulation is created by mixing a test powder, or material, with a liquid, such as water. In this embodiment, the test powder may be a dehydrated test powder that is selected based on the surface or material being tested whereby the water assists to reconstitute the test powder. Depending on the type of test powder, the test powder may become unstable when in a liquid for an extended or predetermined period of time, whereby in some embodiments, the test formulation is created just prior to testing. To increase a stability of the test formulation, other components may be added to the test formulation. In other embodiments, the test formulation may be obtained in a liquid form if the test powder within the test formulation can remain stable in a liquid. In one embodiment, the selected test powder reacts with metals ions as they are leaching out from the surface or material of interest or antimicrobial item. Unlike many current systems, the test formulation of the disclosure does not require any bacterial or culture growth, such that the disclosure may be seen as a non-microbial system and method of determining antimicrobial activity for a surface, material or antimicrobial item of interest.

The test formulation is then applied to the surface or material of interest (102). For ease of understanding, use of the phrase “surface of interest” in the following description also includes a “material of interest”. The test formulation may be applied in any direction, such as to a horizontal surface or a vertical surface.

The reaction between the surface of interest with the test formulation can then be observed (or analyzed) (104). If there is antimicrobial activity (i.e. a leaching out of metal ions or generation of reactive oxygen species) within the surface of interest, the test formulation experiences a visible color change where the level of color change and/or the time it takes for the test formulation to change color provides an indication with respect to the antimicrobial efficacy of the surface of interest. This observation may be performed manually, such as by a user's eyes, or may be automated and performed by machinery, such as, but not limited to, a spectrophotometer or a colorimeter. Part of the observation may include correlating the level and/or speed of color change with a level of antimicrobial activity or the correlation (106) may be performed separate to the observation. In one embodiment, this correlation may be based on comparing the color change with a chart or graph showing color change vs level of antimicrobial efficacy. In another embodiment, the correlation may be to compare the intensity or speed of the color change with predetermined values.

For example, if there is a slow or minimal color change, it may be determined that the surface of interest has a low level of antimicrobial efficacy whereby the color change may serve as an indicator to the user that actions may need to be taken to address the antimicrobial efficacy of the surface of interest. By using the system and method of the disclosure, there is no need for laboratory testing or the time consuming procedure to culture or grow a test bacteria sample. The method and system of the disclosure may be seen as an on-the-spot antimicrobial efficacy test.

In use, the test formulation may be applied to the surface of interest, or antimicrobial item, using different apparatus. Examples of some apparatus are shown in FIG. 1b.

For example, the test formulation may be applied using a stick-on system where the test formulation is applied to a material and then applied to a surface of interest with tape, or the like such that the material with the test formulation is in contact with the surface of interest. In one embodiment, the stick-on system may be used to test vertical surfaces of different geometric shapes. A second apparatus may be seen as a spray-on testing system where the test formulation is sprayed onto a surface of interest. In one embodiment, the spray-on testing system may be used for the testing of a large surface area. A third apparatus may be seen as a drop test apparatus where a drop of the test formulation is applied to the surface of interest. This embodiment may be used to test porous or non-porous horizontal surfaces. Another apparatus may be a contact testing imprinting gel or paper testing apparatus which may be used to determine a homogeneity of a metal coating on a surface of interest.

In one specific embodiment of the disclosure, the disclosure may be used to test the antimicrobial efficacy of copper (Cu), a Cu surface or a Cu impregnated material. It is assumed that the surface, or material, of interest has been impregnated with copper. In one embodiment, the test formulation that is used for the Cu testing is directed at sensing the release of Cu ions upon direct contact such that the test powder or formulation includes a reagent that is able to change color in the presence of Cu ions within a short period of interaction time, such as, but not limited to, a few minutes. In one embodiment, the test formulation, or solution, changes color (transparent to purple) on a surface of interest when in the presence of Cu ions. In another embodiment, the test formulation includes a component that chelates the metal of interest such that a color change is produced (chromogenic chelator).

In another embodiment of the disclosure for copper testing, the embodiment may be seen as a screening tool that exploits, or responds to, the release mechanism of Cu for Cu-based antimicrobial items, or products, to determine the level of leaching of Cu ions from the antimicrobial item, surface or material of interest.

In order to prove and/or research the relationship between the method and system of the disclosure with respect to actual antimicrobial activity, a study was performed to correlate experimental results from the test formulation relating to Cu ion release from solid metallic surfaces and the antimicrobial efficacy of them with a standard Environmental Protection Agency (EPA) test for copper-based antimicrobial hard surfaces.

In summary, the rate at which Cu ions were released from the solid metallic surface being tested correlated well with the reduction of bacteria numbers using Pseudomonas aeruginosa as a model organism. When the test formulation was applied to the surface for testing, there was a color change in the test formulation, or test solution, from transparent to purple. Therefore, it was concluded that the color change intensity of the test formulation could be translated, or correlated, into a level of antimicrobial activity to provide an indication to a tester of the efficacy of the antimicrobial properties of the surface of interest. Results of testing using the method and system of the disclosure may also be used to inform, or provide an indication to, the tester if there is a need to adjust the surface coating or alloying strategy (to increase antimicrobial efficiency) for the manufacture of the item having the surface of interest.

In another embodiment, the disclosure may be seen as a method and system to probe the redox state of Cu ions from the total number Cu ions by eliminating or removing a reducing agent, and therefore measuring the relative amounts of Cu¹⁺ versus Cu²⁺.

To determine test powder, formulation, or solution components, testing focused on determining a method to sense the release rate of Cu ions directly on the surface of interest. In this test phase, the testing was performed in an aqueous test solution. Through testing, it was determined that the test formulation should include a chelator of Cu₊₁ ions such as, but not limited to, bicinchoninic acid (BCA) which forms a dark purple color in the alkaline environment and is frequently used to quantify proteins. Other chelators include, but are not limited to, EDTA, glutathione, ammonia, amine, imine complexes, cuprizone and Folin.

To understand the cumulative role of Cu ions regardless of the oxidation state of the Cu ions, all the Cu²⁺ produced by the surface of interest are required to be converted into Cu¹⁺, so the BCA could chelate with Cu⁺¹. In testing, several attempts were made with different concentrations of weak reducing agents that were able to reduce Cu⁺² to Cu⁺¹. In one specific embodiment, ascorbic acid at 10 mM was selected, although others are contemplated such as, but not limited to, dextrose (or similar reducing sugars), antioxidants, oxalic acid or Vitamin E.

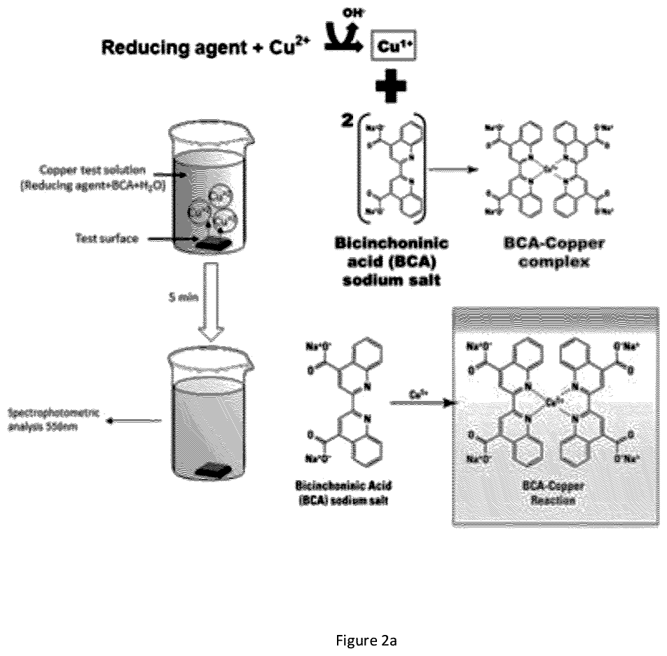

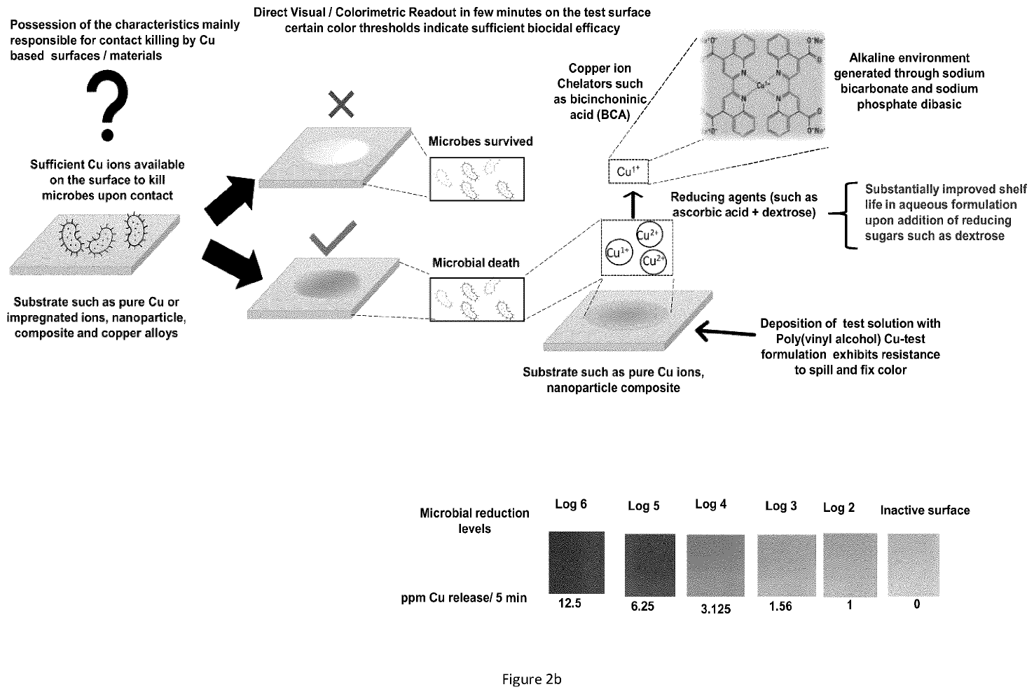

Using BCA and ascorbic acid in the test formulation, testing of the surface of interest relies on two reactions between the Cu and the test formulation. The first reaction is based on the redox reaction of Cu where Cu²⁺ forms Cu¹⁺ in the presence of the ascorbic acid. Next, two molecules of BCA chelate with each Cu⁺ ion, forming a purple-colored product that strongly absorbs light at a wavelength of 558 nm. Different reactions are schematically shown in FIGS. 2a and 2b. As can be seen in the bottom of FIG. 2b, one correlation of antimicrobial activity and level (shade/intensity) of color change is shown.

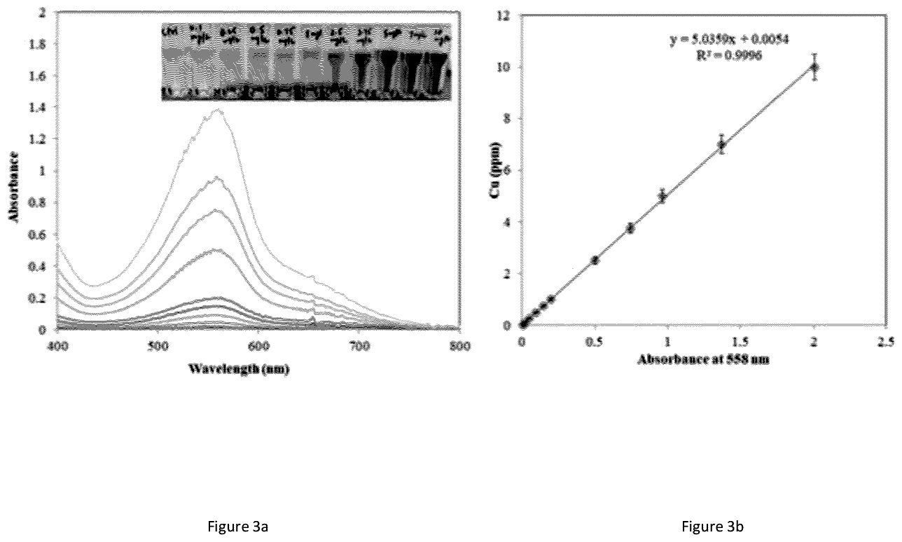

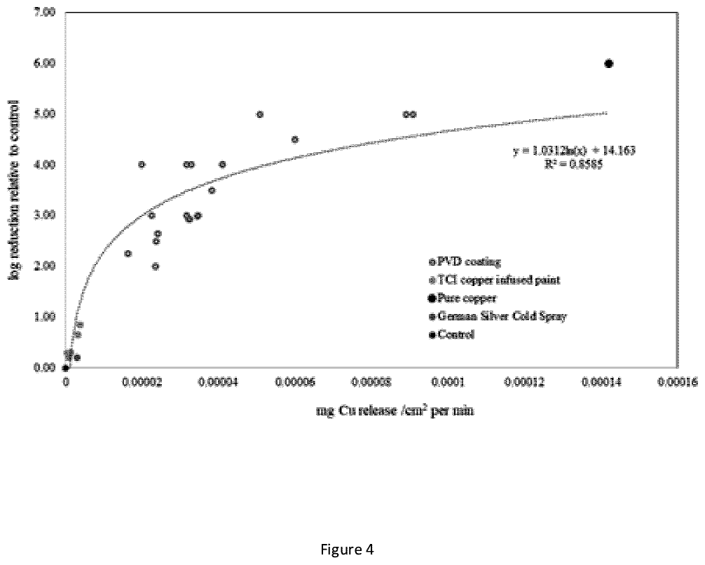

A further test showed that only Cu⁺¹ was formulated if the test formulation did not include a reducing agent. It was inferred that by simply subtracting the amount of Cu¹⁺ ions from the total number of Cu ions would provide or produce the number of Cu²⁺ ions. The reaction mixtures prepared to determine Cu ions were used to produce a standard curve by using different concentrations of CuCl₂ and CuCl in solution. The BCA copper assay was linearly measuring copper concentration in the range of 0.1-10 ppm with the R² value of 0.99 as shown in the charts of FIGS. 3a and 3b. A cross-reactivity test indicated that only Cu-based surfaces were responsive to the test solution or formulation including BCA and ascorbic acid.

For copper testing, one specific embodiment of a test formulation may include 10 mM each of BCA and ascorbic acid and 50 mM of sodium bicarbonate and 10 mM of sodium phosphate dibasic. In another embodiment, the test formulation may include BCA, ascorbic acid, sodium bicarbonate, sodium phosphate dibasic, dextrose and poly(vinyl alcohol).

For experiments, extra pure water with a specific resistivity 18 MO cm was used to dissolve the test powder or material. The test or experimental surfaces or materials were copper, copper-based alloys, physical vapor deposition (PVD) and Cu infused painted and stainless-steel surfaces called “coupons”. Prior to antimicrobial testing, the coupons were placed in 1% detergent solution (Liquinox) for 2-4 hr to degrease and then rinsed thoroughly with deionized water and allowed to dry. The coupons were then soaked in 95-98% ethanol for 5 to 10 minutes to decontaminate. The coupons were removed with sterile forceps and placed in or on a sterile petri dish to dry in a biosafety cabinet overnight under sterile conditions.

Direct Measurement of Copper Ions from the Surface

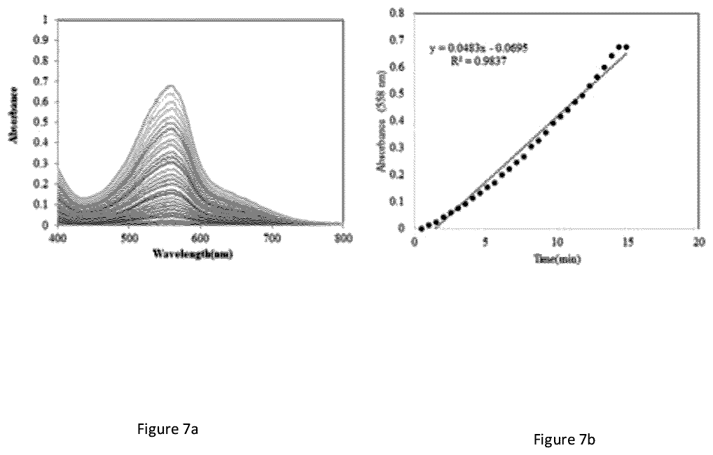

Testing of the coupons was then performed by producing or obtaining a test formulation for use in determining antimicrobial efficacy of the copper coupons (approximately 1 cm×1 cm) or for providing an indication of antimicrobial efficacy of the copper coupons. For the experiments, the test formulation or solution included 10 mM each of the BCA and ascorbic acid, 50 of mM sodium bicarbonate and 10 of mM sodium phosphate dibasic mixed in water. For the current experiment, copper ions released into the aqueous phase by the coupons were tested by the test formulation with a resulting color change being able to be measured or correlated to provide a numerical indication of the antimicrobial activity such as shown in FIGS. 7a and 7b.

After generating, or obtaining, the test formulation, 1 mL of the test formulation was applied to each of the coupons, covering a surface area of approximately 1 inch×1 inch. The applied solution was given different exposure times and the visual color change observed during these exposure times. The exposure time was recorded for the different individual samples with the help of a stopwatch matching, or recording, color change intensity with the time elapsed. The remaining test solution was recovered from each coupon and analyzed by a spectrophotometer which provided numerical results for correlation, such as with the graphs of FIGS. 7a and/or 7b.

For a spectrophotometer test of the pure copper coupon, the pure copper coupon was cut into a 1×1 cm square and placed inside a cuvette with the test solution to check or observe the release rate of Cu ions in real time over a period of 15 minutes. The assay was performed in two distinct ways using a test formulation with ascorbic acid (10 mM) and a test formulation without ascorbic acid to determine the amount of total copper and Cu¹⁺ ions released from, or present on, the pure Cu surface. Subtracting the number of Cu⁺¹ ions from the total number of copper ions provided the amount of Cu²⁺ released from the surface. A standard curve was obtained by taking 1 mL of different concentrations (0-100 ppm) of CuCl and CuCl₂ that interacted with 1 mL of the test solutions independently. The absorbance was recorded at 558 nm. The evolution of the peak at 558 nm clearly indicated that the test solution worked directly on the surface of interest. The regression analysis of the maximum, or high, absorbance at 558 nm versus time yielded a R² value of 0.98. The response of the color change obtained from the leach out of copper ions directly from the surfaces was derived from an equation obtained from linear regression analysis of the standard curve (FIG. 3b).

Further experiments were performed by placing 100 μL of the test formulation or solution directly on the Cu coupons. A color change of the test solution was observed over time. The response of the test solution was highest on the pure copper coupon followed by Cu—Ni PVD (physical vapour deposition) surface and a copper-infused painted surface. The control surfaces without any residual copper indicated no change in color. The same pattern existed in their antimicrobial performance.

Surface Imprinting

In order to mark the areas of the surface that was able to leach out copper ions, surface imprinting was used. This information provides insight with respect to if the copper particles, or ions, are properly exposed and homogenously distributed all over the surface of interest to confirm a consistent antimicrobial performance over the entire surface of interest. This may also be used as a test to determine the antimicrobial activity level. Along with use of agarose gel, the surface imprinting may also be performed using papers or other materials that are impregnated with the test formulation.

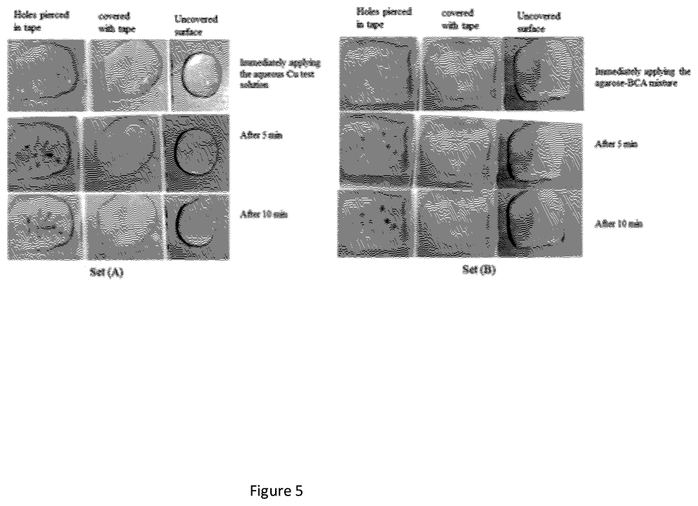

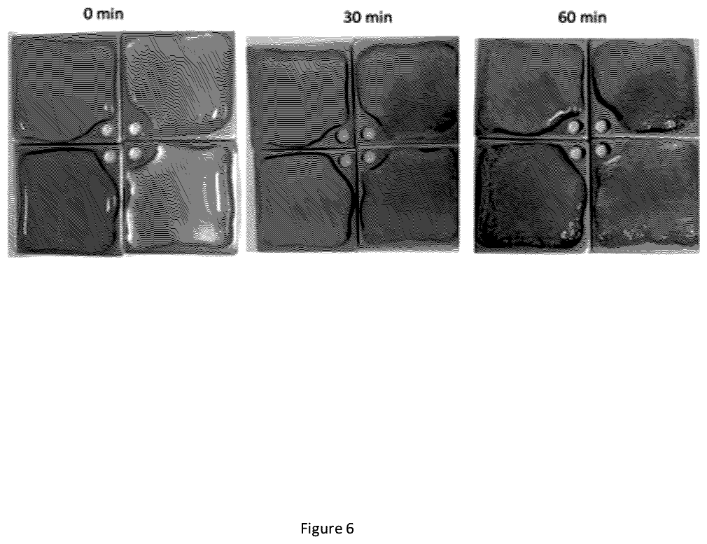

A 3% agarose gel was prepared in deionized water. The gel was melted by heating. In the molten agarose gel, the copper test solution was added in a 1:1 ratio. Test surfaces, or coupons, were prepared with tape having holes on top of the surfaces. The thin layer of molten gel was poured on the test surfaces directly. When the Cu surface came in contact with the gel, the agarose-BCA gel started to evolve to a purple color according to the pattern on the tape. The colored pattern remained intact even after one hour. The spots releasing Cu ions were visualized on the real surfaces (FIG. 5) and the real paint samples (FIG. 6). These surfaces were created by embedding copper particles in paint on the test surfaces. The BCA-agarose gel was placed on the different surfaces and allowed to sit for 60 minutes. Within the first 5 minutes of contact time, a visible color change started to develop in the areas releasing Cu ions which got darker over the period of 30 min. It can be clearly seen in FIG. 6 that the uncovered spots were changing color but the color started to spread within 5 min. The quick diffusion of the color in the surrounding made it difficult to recognize the area actually releasing Cu ions.

In another embodiment, the disclosure includes an embodiment of testing antimicrobial efficacy for a vertical surface. In this embodiment, the test formulation includes BCA, ascorbic acid, sodium bicarbonate, sodium phosphate dibasic, dextrose and poly(vinyl alcohol) where the poly(vinyl alcohol) acts as a viscosity modifier which may assist to slow or stop dripping of the test formation when applied to the vertical surface.

In a specific experiment, 200 ul test formulation was used to soak a piece of gauze which was then taped to a vertical surface. A color change was observed on the gauze and intensity of the color change analyzed (and/or correlated) to determine the antimicrobial efficacy of the vertical surface.

These results indicated that application of the test formulation to a copper surface using this surface imprinting technique enabled a user to observe a color change of the test formulation indicating antimicrobial efficacy. This also allowed a user to visualize the phenomena happening directly on the surface.

In a further embodiment, the system may include a spectrophotometer or the like to measure a rate of metal ion release in real time. Along with the visible change in color and the change rate in the color, an appearance or intensity of the color change may be translated or correlated into antimicrobial performance.

As discussed above, the experimental results using the test formulation were correlated with results using standard bacterial and fungal species using standard microorganisms, or current testing. In one embodiment, the release rate of metal ions from the surface of interest in both test scenarios may be used. This co-relationship may be used to develop the algorithms to predict the possible antimicrobial action. A mathematical model may be developed to predict the life of the antimicrobial-coating under different conditions.

In another embodiment, the method and system relies on the direct measurements of the released ‘active Cu ions’ from the surface which are actually bioavailable and participate in the contact killing. As discussed above, this measurement of release may be used for predicting or indicating antimicrobial efficacy.

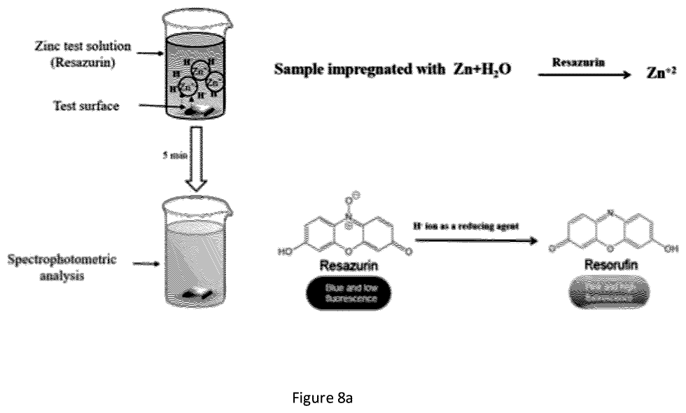

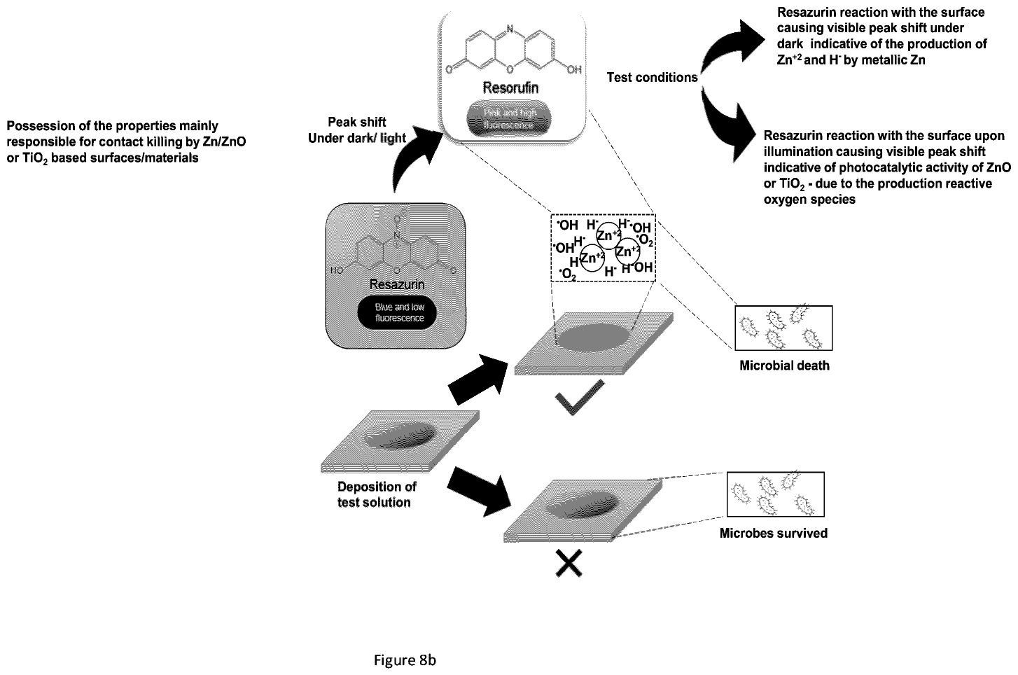

In another embodiment, the system and method of the disclosure may be used to determine the antimicrobial efficacy of a zinc surface, or a material impregnated with zinc and provides a non-microbial prediction, or indication, of antimicrobial efficacy of the zinc surface or material of interest being tested. In one embodiment, the test formulation may be applied to the zinc surface and, based upon the chemical behavior of the Zn and ZnO (other photocatalytic film) alone or in combination, with the test formulation, an approximate determination or indication of the antimicrobial efficacy of the zinc surface can be provided or observed and then correlated. In one embodiment, the disclosure provides results that can be translated into antimicrobial efficacy by simply observing and/or correlating the color change over a period of time. Schematic diagrams of embodiment of testing a zinc surface are schematically shown in FIGS. 8a and 8b.

In one embodiment, the test formulation or solution includes resazurin which is a redox-sensitive dye and has a blue to purple color that can be irreversibly reduced, or changed, to a pink-colored and highly fluorescent resorufin. Instead of using resazurin, other redox dyes, such as, but not limited to, 2,3-Bis-(2-Methoxy-4-Nitro-5-Sulfophenyl)-2H-Tetrazolium-5-Carboxanilide (XTT), nitro blue tetrazolium (NBT), 3-(4,5-dimethylthiazol-2-yl)-2,5-diphenyltetrazolium bromide (MTT) and 5-Cyano-2,3-ditolyl tetrazolium chloride (CTC) are contemplated.

As can be seen in FIG. 8a, a zinc test surface is placed in a beaker, or container, containing the test formulation or solution that includes the redox-sensitive dye (such as, but not limited to, resazurin). After a predetermined period of time, if there is antimicrobial activity, the test solution changes color which can enable a user to relate the rate of change of color with the antimicrobial performance of the Zn/ZnO surface of interest. A similar schematic is shown in FIG. 8b.

In one embodiment for the method and apparatus for testing a zinc surface, the disclosure, or test formulation, includes redox dyes that are able to probe the photocatalytic and redox activity of these zinc coatings simultaneously. In one specific embodiment of a test formulation, extra pure water with a resistivity of 18 MO cm may be mixed with resazurin.

For experiment purposes, pure metallic Zn foil was cut to a suitable coupon size and zinc and zinc oxide powder (200 mesh) was also used. Prior to antimicrobial testing, the coupons were placed in 1% detergent solution (Liquinox) for 2-4 hr to degrease, then rinsed thoroughly with deionized water and allowed to dry. The coupons were soaked in 95-98% ethanol for 5 to 10 minutes to decontaminate. The zinc coupons were removed with the help of sterile forceps and placed in or on a sterile petri dish to let them dry in a biosafety cabinet overnight under sterile conditions. To prepare the ZnO coated surface, 2 g of the zinc oxide powder was added to 50 ml of deionized water and coated on a plastic petri dish with help of a brush and left to dry under sterile conditions. 10 mg of metallic zinc was sprinkled on the wet ZnO coated surface (75 cm²) and dried.

Measurement of Contact Killing

To assess contact killing, a version of the EPA protocol for copper-based antimicrobial surfaces was used such as discussed above.

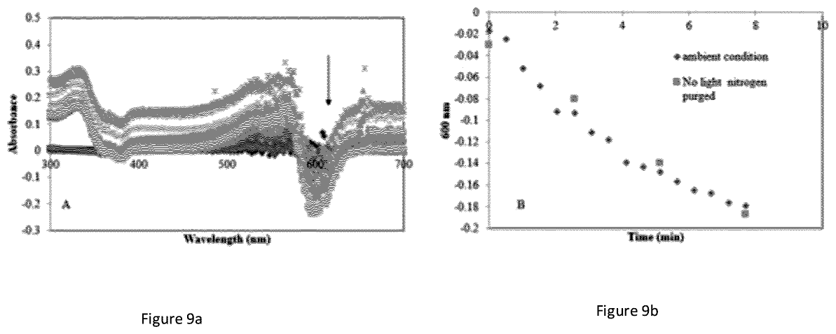

In some further experiments, the reducibility of the Zn was probed using resazurin as an electron acceptor and was conducted in ambient, dark and N₂ purged conditions to eliminate or reduce the role of light and oxygen. Identical reduction rates were recorded under both conditions as shown in FIG. 9 where FIG. 9a represents the whole spectral scan while FIG. 9b represents the evolution of the resazurin peak at 600 nm over time.

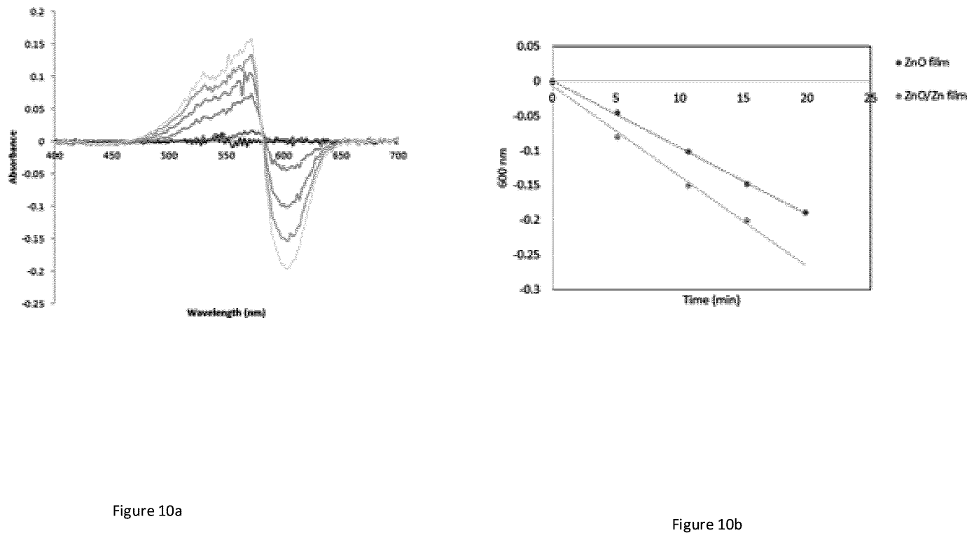

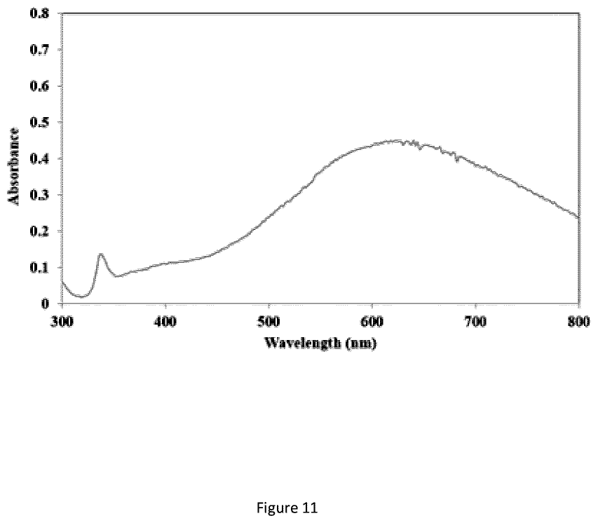

In further testing, the reaction between the pure Zn coupon and the test solution resulted in a color change in the test solution without any external stimulus while there was no color change with the ZnO coupon and the test formulation. From this, it was concluded that that Zn metal can reduce resazurin without depending upon oxygen or illumination but a ZnO film is not able to reduce resazurin without the presence of external stimulus but showed a color change when illuminated. The rate of photocatalysis was improved (as shown in FIG. 10) when the ZnO film was conjugated with a trace amount of Zn. FIG. 10a shows the photocatalytic conversion of resazurin over the whole spectral scan while FIG. 10b shows an evolution of resazurin peak at 600 nm over time.

In experiments, the bactericidal activities of the samples were evaluated by the inactivation of Pseudomonas aeroginosa, on the basis of the decrease in the number of colony forming units of Pseudomonas aeroginosa recorded on the agar plate. According to FIGS. 9a and 9b, the survival rate for Pseudomonas on the ZnO films with 2 and 4 h under UV illumination decreases up to 50 to 100% respectively relative to control. The survival of bacteria on the plastic plate without ZnO films and under UV illumination alone does not change. The pure zinc plate with or without illumination was able to reduce viable bacteria only up to 70% percent respectively. However, when Zn/ZnO conjugated film was used, 100% inactivation was observed after 2 hours under ambient conditions with and without UV illumination. The improved performance of the Zn/ZnO conjugated system may be attributed to electrons generated from Zn which can be transferred to the conduction band of ZnO in an energetically favorable way. This experiment clearly shows the increase in the reduction of the resazurin can be related to the enhanced antimicrobial action.

In another embodiment, the system and method of the disclosure may be used to determine antimicrobial efficacy of a silver surface. The antimicrobial performance of a product is directly linked to the rate of release of silver (Ag⁺) ion per unit area of the area of the product treated with silver and therefore a test formulation directed at producing a color change when in contact with leaching silver ions was developed.

It was shown in experiments that the chromogenic detection of silver ions can be performed using controlled seed growth of silver nanoparticles, more specifically prismatic silver nanoparticles although other shaped nanoparticles are contemplated. A principle behind the detection of Ag⁺ ions leaching out from the surface or material is to convert them back to colloidal silver with the help of a reducing agent. The presence of prismatic silver nanoparticles in the test formulation allows the reaction to happen immediately at room temperature even in the presence of a weak reducing agent in a short period of time. Since the surface plasmon resonance band depends strongly upon the size and shape of nanoparticles, the controlled growth of silver nanoparticles during the reaction produces a visible darkening of the color with a blue shift or tint.

A bioavailable concentration of silver ions may be used to build a relationship between the amount of silver ions released from the surface of interest with the antimicrobial performance of the surface of interest. As with the other embodiments, after applying the test formulation to the silver surface of interest, a handheld measuring device may be used to quantify the amount of silver that is leaching to provide an indication to a user of the antimicrobial efficacy of the products based upon the amount of Ag⁺ ions being released from the product or surface of interest. This quantification may also be used to provide, or correlate, a relationship between the level of color change, rate of color change and the level of antimicrobial activity. As the rate of release of Ag⁺ ions per unit area is related to their biocidal action, the system and method of the disclosure may provide an indication or assistance to a user to quickly estimate, or understand, the biocidal performance of the surface being tested before sending it out for any further verification by a third party.

In the current embodiment, the test solution or formulation includes citrate, or PVP, capped silver nanoparticles, such as nanoprisms. In one embodiment, prismatic silver nanoparticles are chosen based on their high extinction coefficient due to their anisotropic morphology, compared to spherical or quasi-spherical silver nanoparticles, however, any shaped nanoparticles may be used.

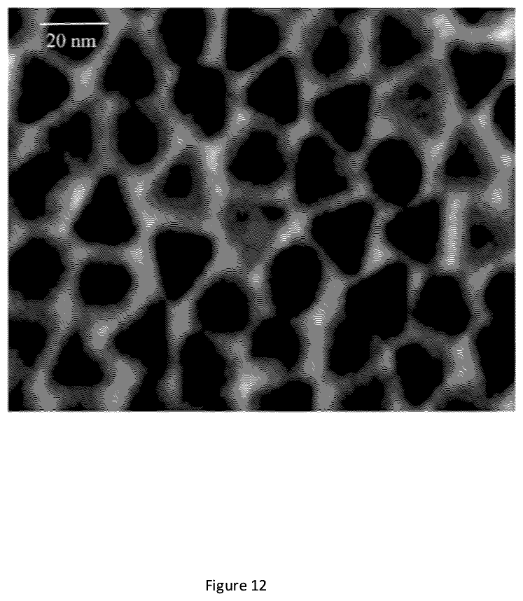

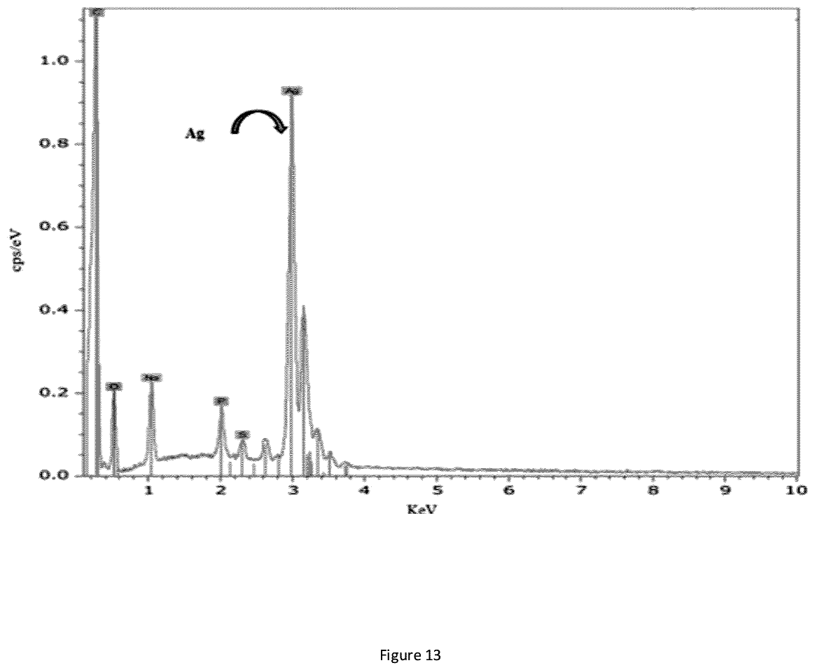

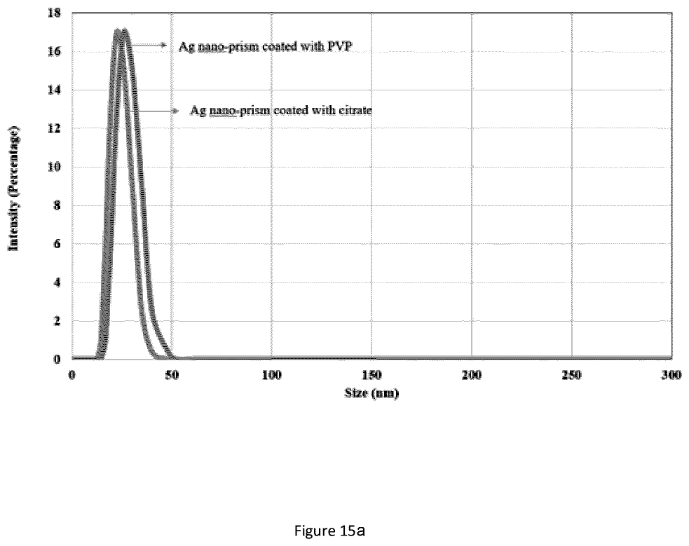

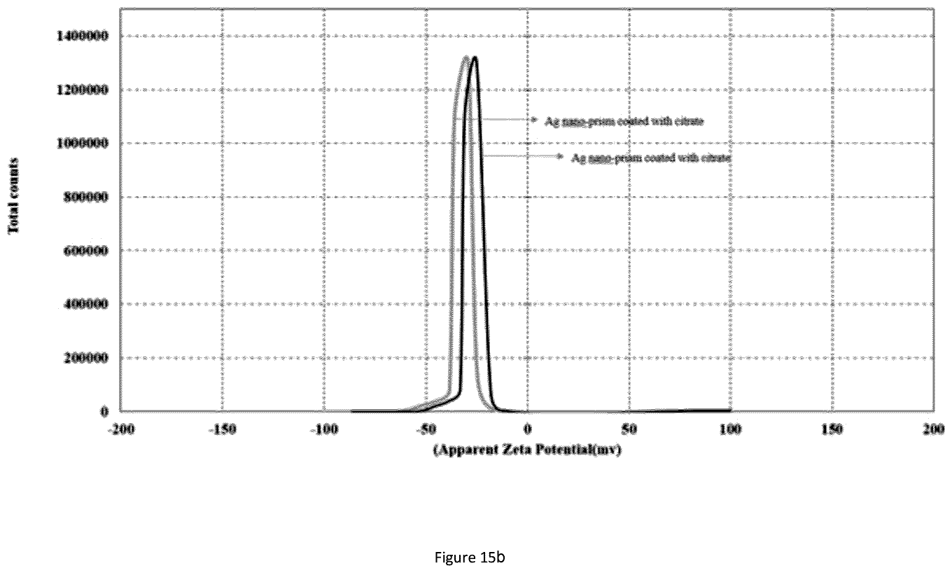

In experiments, prepared silver nanoprisms were analyzed by UV-visible spectrophotometer (FIG. 12), transmission electron microscopy (FIG. 13), energy dispersive x-ray spectroscopy (EDX) (FIG. 14), dynamic light scattering (FIG. 15a) and zeta potential measurements (FIG. 15b). One advantage of using silver nanoprisms is their high stability. In one embodiment, to improve the reaction of silver nanoprisms in the test solution, the silver nanoprisms are shielded from aggregation and degradation while maintaining their excellent plasmonic properties. As such, in one embodiment, the silver nanoprisms were coated with PVP with passivated shells to protect them from etching or other effects. The zeta potential measurements (FIG. 15b) of sodium citrate capped silver nanoprism changed from −29.28 mV to −25.7 showed that the PVP had indeed capped the nanoparticles' surfaces and contributed to the electrostatic stability. The influence of pH and ionic strength on the stability of the prismatic silver nanoprisms was also tested and no noticeable SPR peak shifts were observed upon the addition of buffers with different pH range and concentrations.

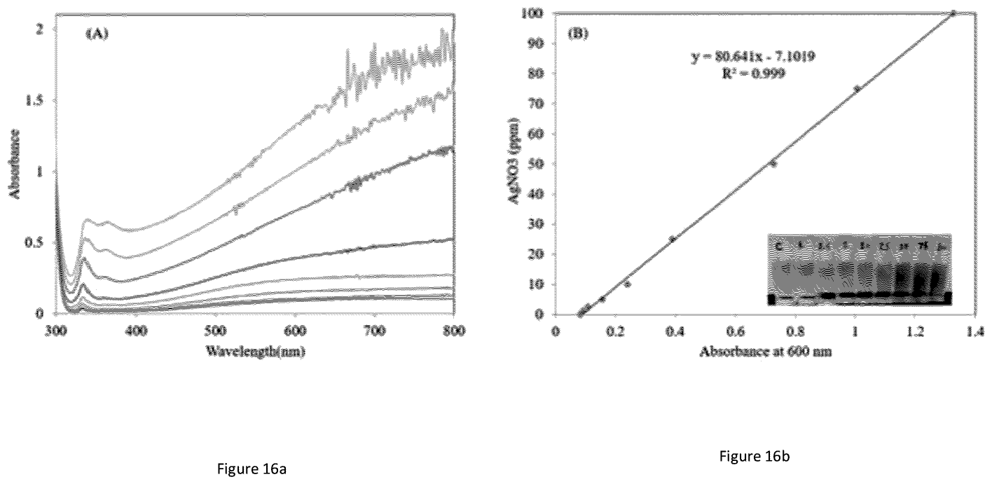

FIGS. 12 and 16 are the UV-vis spectra before and after the reduction of Ag⁺ indicated the peaks around 630 nm were the characteristic surface plasmon resonance (SPR) absorption of Ag nanoprism; whereas as the further broadening of the peak at 700 nm was the indication of the growth of the nanoprism in the presence of Ag⁺. The rapid formation of Ag shell on the surface of silver nanoprism can be attributed to the presence of Ag nanoprism initiated the formation of Ag clusters containing several Ag atoms by decreasing the redox potential of Ag⁺/Ag couple, speeding up the nucleation and deposition. Therefore, Ag shell can be easily formed on the surface of Ag-nanoprism. The darkening of the color of silver-prism can be seen upon the addition of different concentrations of silver nitrate. The Ag-prism assay for Ag⁺ ions was linearly measuring Ag⁺ concentration in the range of 0.1-100 ppm with the R² value of 0.99 (FIG. 17). It seems that, beyond 100 ppm of AgNO3, the edge of the prismatic nanoparticles started to round up and produce greenish yellow color due to the presence of excessive amount silver ions present in the solution.

In some embodiments, the silver nanoprisms may enable the identification of silver ions by further provoking the weak reducing ability of ascorbic acid where the silver nanoprisms act like a catalyst and seeds for the reduction of Ag+ ions on the surface of Ag nanoprism by rapidly depositing a silver shell around the nanoprism leading to darkening of the color, whereas this reaction, in the absence of Ag nanoprism, is not able to produce a visible color change.

In one embodiment, the production of citrate capped silver nanoprisms includes silver nanoprisms, 100 μL of silver nitrate (100 mM), 100 mM of trisodium citrate (1.5 mL), and 280 μL of 30% hydrogen peroxide that was mixed together and diluted to 100 mL with water in a flask. Other methods of manufacturing PVP or citrate silver nanoparticles, such as silver nanoprisms are known and contemplated. Use of PVP or citrate capped gold nanoparticles are also contemplated. In another embodiment, the test formulation may include a component that changes color when in the presence or in contact with silver ions.

The solution was vigorously stirred for 10 min, and 1 mL of 0.1 M sodium borohydride was rapidly added. After 2 min, the colorless solution turned yellow and then rapidly darkened until a stable blue color was developed after approximately 5 minutes. The nanoprisms were further capped with 0.1% PVP. The resulting suspension was stored in the dark at 4° C. This procedure allowed for the silver nanoprisms to be easily synthesized on a large scale with a degree of high stability.

For experiments, the following materials were used. Silver nitrate (AgNO₃, 99.99%), trisodium citrate dihydrate (C₆H₅O₇Na₃·2H₂O, 99.99%), sodium borohydride (NaBH₄, 99.99%), hydrogen peroxide (H₂O₂ 30%), HNO₃, and polyvinylpyrrolidone (PVP; MW=40,000). The test samples were cloth tethered with silver. All of the solutions were prepared in ultrapure water with resistivity 18 MO cm⁻¹. Piranha solution (30:70 v/v solution of 30% hydrogen peroxide and concentrated sulfuric acid) was used for cleaning of glassware.

To test the test formulation to confirm its efficiency for silver ion sensing, 200 μL of a test, or Ag-prism (OD 1 at 630 nm), solution and 100 μL of ascorbic acid (100 mM) was added to 2 ml of different concentrations of AgNO₃ (0-0.1 ppm respectively). These mixed solutions were agitated slowly for 10 min at room temperature before their UV-vis spectra were determined. The nanoprism stability was determined at a very broad pH range, from 5 to 7.5, and phosphate buffer concentration of 10-2 M.

To test the test formulation to confirm its efficiency for silver ion sensing from a cloth sample, a silver treated cloth was cut into 10 cm×10 cm pieces. The pieces of cloth were placed in 50 ml of de-ionized water for 1 hour. The leach out of the silver ions was tested by using 200 μL of the test, or Ag-prism (OD 1), solution, 100 μL of ascorbic acid (100 mM) called “assay solution”, and different 2 ml of leach out solution.

In using the test formulation to assess the antimicrobial activity of the silver tethered cloth, to assess contact killing, a modified version of the EPA protocol was used for a cloth antimicrobial test. For the current experience, the bacteria used was Pseudomonas aeruginosa ATCC 9027. The Pseudomonas culture was inoculated in sterile Cetramide media and incubated for the period of 12 hours. After the cultivation, the suspension was centrifuged and washed twice using 10 mM of sterile PBS pH7.4. The turbidity of the bacterial concentration was adjusted in the range of 10⁸ cfu/mL with the help of a UV visible spectrophotometer (600 nm at the optical density of 0.2). The concentration of the viable bacterial cells was duly verified using heterotrophic plate method after preparing appropriate dilutions of the bacterial sample. The test cloth (1×1 inch) is placed inside a 50 ml beaker and then carefully applied of 20 μL of 10⁸ cfu/mL of the culture all over the surface and placed at room temperature for the period of 1 hours (EPA, 2009). Then, 20 mL of sterilized PBS was added in the beaker and sonicate for 5 minutes for recovery of P. aeruginosa from the cloth. The recovered solution was further diluted at three levels of tenfold dilutions and plated 0.1 mL using plate count agar. Plates were incubated at about 37° C. for 24 hours.

It was determined that the antimicrobial efficacy of silver relies on the sustained oxidation and release of very small quantities of silver cations (Ag⁺) into an aqueous environment or an environment with moisture. Silver cations strongly interact with thiol groups (—SH) in biomolecules. In bacteria, this causes the inactivation of enzymes in the outer membrane, which disturbs its permeability and the proton motive force leading to pits in the membrane and eventual cell death.

In one embodiment, components for the system or test formulation may be selected or based on the microbial load reduction ability of the silver-containing materials based on the in-situ release rate of Ag ions measured with a reagent able to change color in the presence of Ag⁺ ions within a short period of time. The test formulation provides an estimated silver ions concentration (such as via color change) in the samples using the seed mediated growth of prismatic nanosilver particles which may be correlated to antimicrobial activity.

In one experiment, a study was conducted to determine a release rate by immersing a 10×10 cm cloth in a beaker containing 50 ml of the deionized water. The reaction was recorded after 1 hour and then 2 ml samples were taken from the beaker. The samples showed immediate color change upon interaction with the test formulation including citrate capped silver nanoprisms and ascorbic acid). The response of the color change obtained from the leach out of Ag⁺ ions from the cloth was derived from an equation obtained from linear regression analysis of the standard curve from FIG. 16b. The release rate also allows for the calculation of longevity of the treated cloth after repeated washings.

A second part of the study was dedicated to determining the antimicrobial activity using the bacterium P. aeruginosa on the same cloth used in the release rate study. To verify that the Ag release rate correlated with the decrease in viable bacterial count in the assay, P. aeruginosa viability was analyzed after 1 hours of exposure. Several independent experiments were carried out to determine the reduction of P. aeruginosa cells on the cloth for contact killing according to EPA standards for the testing of copper surfaces and alloys (EPA, 2009). The release rate of silver obtained from the different clothes were plotted against the log reduction of the concentration of the P. aeruginosa. Results obtained from 6 cloth pieces were in good correlation (R²=0.87) indicating that measurement of Ag ion release rate per unit area of the cloth is a feasible analysis strategy. The mathematical equation obtained may be used to predict the efficacy of unknown samples without handling the pathogenic bacteria by simply comparing the release rate.

In another embodiment, the disclosure may include an apparatus that may predict the antimicrobial efficacy and longevity of the treatment based on release rate, of leaching, parameters. An advantage of the disclosure is the provision of a system and method to quickly quantify antimicrobial capability of the surface based on the intensity of the color change of the solution.

In the preceding description, for purposes of explanation, numerous details are set forth in order to provide a thorough understanding of the embodiments. However, it will be apparent to one skilled in the art that these specific details may not be required. In other instances, well-known structures are shown in block diagram form in order not to obscure the understanding.

The above-described embodiments of the disclosure are intended to be examples of the present disclosure and alterations and modifications may be effected thereto, by those of skill in the art, without departing from the scope of the disclosure.

What is claimed is:

1. A method for non-microbial prediction of antimicrobial efficacy of an antimicrobial item comprising:
producing a test solution;
applying the test solution to the antimicrobial item;
analyzing a color change of the test solution after exposure of the test solution to the antimicrobial item; and
correlating the color change with a level of antimicrobial efficacy.

2. The method of claim 1 wherein the test solution reacts with a leaching of metal ions from the antimicrobial item or generation of reactive oxygen species on the antimicrobial item.

3. The method of claim 1 wherein producing a test solution comprises mixing bicinchoninic acid (BCA) ascorbic acid, sodium bicarbonate and sodium phosphate dibasic.

4. The method of claim 1 wherein the antimicrobial item comprises a copper surface, copper based alloys, copper nanoparticles or copper ion composites.

5. The method of claim 1 wherein producing a test solution comprises mixing resazurin with a liquid.

6. The method of claim 5 wherein mixing resazurin with a liquid comprises mixing resazurin with water.

7. The method of claim 5 wherein the antimicrobial item comprises a zinc surface.

8. The method of claim 1 wherein producing a test solution comprises mixing, silver nitrate, trisodium citrate, sodium borohydride and hydrogen peroxide to create silver nanoparticles.

9. The method of claim 8 wherein the silver nanoparticles are silver nanoprisms.

10. The method of claim 8 wherein the antimicrobial item comprises a silver surface.

11. A test material for use in non-microbial prediction of antimicrobial efficacy of an antimicrobial item comprising:
a component that changes color when in contact with metal ions leaching from the antimicrobial item or reactive oxygen species on the antimicrobial item.

12. The test material of claim 11 wherein the component comprises bicinchoninic acid (BCA).

13. The test material of claim 12 wherein the test material further comprises:
a weak reducing agent.

14. The test material of claim 13 wherein the test material further comprises:
at least one of ascorbic acid, sodium bicarbonate, sodium phosphate dibasic, dextrose and poly(vinyl alcohol).

15. The test material of claim 11 wherein the component comprises a redox dye.

16. The test material of claim 15 wherein the redox dye comprises one of resazurin, 2,3-Bis-(2-Methoxy-4-Nitro-5-Sulfophenyl)-2H-Tetrazolium-5-Carboxanilide (XTT), nitro blue tetrazolium (NBT), 3-(4,5-dimethylthiazol-2-yl)-2,5-diphenyltetrazolium bromide (MTT) or 5-Cyano-2,3-ditolyl tetrazolium chloride (CTC).

17. The test material of claim 11 wherein the component comprises citrate capped silver nanoparticles, PVP capped nanoparticles, citrate capped gold nanoparticles or PVP capped gold nanoparticles.

18. The test material of claim 17 wherein the silver nanoparticles comprises prismatic silver nanoparticles.
